(19) United States
(12) Patent Application Publication
(10) Pub. No.: US 2005/0287192 A1
(43) Pub. Date: Dec. 29, 2005

(54) Wound bandage comprising a non-enzymatic antioxidant

(76) Inventors: Hakan Nygren, Billdals (SE); Herman Sahlin, Skytteskogsgatan (SE)

Correspondence Address: YOUNG & THOMPSON, 745 SOUTH 23RD STREET, 2ND FLOOR, ARLINGTON, VA, 22202, US

(21) Appl. No.: 10/519,622

(22) PCT Filed: Jun. 27, 2003
(86) PCT No.: PCT/SE03/01131

§ 371 Date: Aug. 30, 2005

(30) Foreign Application Priority Data
Jul. 3, 2002 (SE) 0202081-6

(51) Int. Cl. A61K 38/05; A61K 31/198; A61L 15/00

(52) U.S. Cl. 424/445; 514/18; 514/562

(57) ABSTRACT

This invention relates to a wound bandage. According to the invention a non-enzymatic low molecular thiolic antioxidant, such as glutathione or N-acetylcysteine, is added to a layer of the wound bandage which, when the bandage is used, comes into contact with a wound.

### TECHNICAL FIELD

This invention relates to a wound bandage.

### BACKGROUND TO THE INVENTION

Lipid peroxidation arises in wound tissue when there is contact between membrane lipids and oxygen or reactive oxygen radicals, such as O₂—. These oxygen radicals are mainly produced by leukocytes and are needed in the defence against bacterial infections but they have the disadvantage that they also damage the body's own cells. Lipid peroxidation products, such as malonaldehyde, 4-hydroxyalkenals, alkanals and alk-2-enals are toxic to leukocytes and prevent the activity of these cells in wound healing. From Ortolani, Conti et al, “The effect of Glutathione and N-Acetylcysteine on Lipoperoxidative Damage in Patients with Early Septic Shock”, American Journal of Respiratory and Critical Care Medicin, Vol 161, pages 1907-1911, it is known how to inject glutathione and N-acetyl-cysteine in patients with early septic shock in order to prevent hyperproduction of free oxygen radicals. From EP-A2-0 945 144 it is known how to use superoxide dismutase, catalase, glutathione peroxidase, myeloperoxidase and enzyme mimics in wound bandages in order to convert reactive oxygen radicals to water and oxygen gas. One disadvantage with such a bandage is that it is technically difficult to work with enzymes as they can easily be destroyed during the production process.

It is the object of this invention to produce a wound bandage which counteracts lipid peroxidation without affecting the activity of the inflammatory cells, e. g. their ability to form oxygen radicals and ability to kill bacteria.

### SUMMARY OF THE INVENTION

This object is achieved according to the invention by means of a wound bandage with added low molecular enzymatic thiolic antioxidants, such as N-acetylcysteine and glutathione, which are more effective than enzymatic antioxidants and technically easier to use. Such antioxidants are added to a layer of the wound bandage which when the bandage is used comes into contact with a wound. These low-molecular-weight additives reduce the occurrence of lipid peroxidation and thus protect the body's own cells without reducing the formation of reactive oxygen. Low-molecular-weight non-enzymatic antioxidants are also more effective than enzymatic antioxidants and technically easier to use.

In a first preferred embodiment a non-enzymatic thiolic antioxidant is added to a wound pad of fibre or foam material.

In a second preferred embodiment the bandage comprises a layer of a hydrophobic or hydrophilic gel, to which a non-enzymatic thiolic antioxidant is added.

### LIST OF FIGURES

The invention will now be described with reference to appended figures, of which;

FIG. 1 and 2 show a bar chart of stress activation of leukocytes in contact with a cotton wool compress with and without additives,

FIG. 3 shows a bar chart of stress activation of leukocytes in contact with a cotton wool compress with addition of glutatione,

FIG. 4 shows a bar chart of the ability of leukocyte cells to be activated by zymosan after being in contact with cotton wool compresses with and without additives,

FIG. 5 shows a bar chart of lipid peroxidation in a leukocyte membrane in contact with a cotton wool compress with and without additives

FIG. 6 shows a bar chart of the ability of leukocytes to kill bacteria in a buffer with and without additives, and

FIG. 7 shows schematically a cross-section through a wound bandage according to an embodiment of the invention.

### DESCRIPTION OF EMBODIMENTS

The effect of cotton wool compresses without and with additives on leukocytes was studied in the following manner.

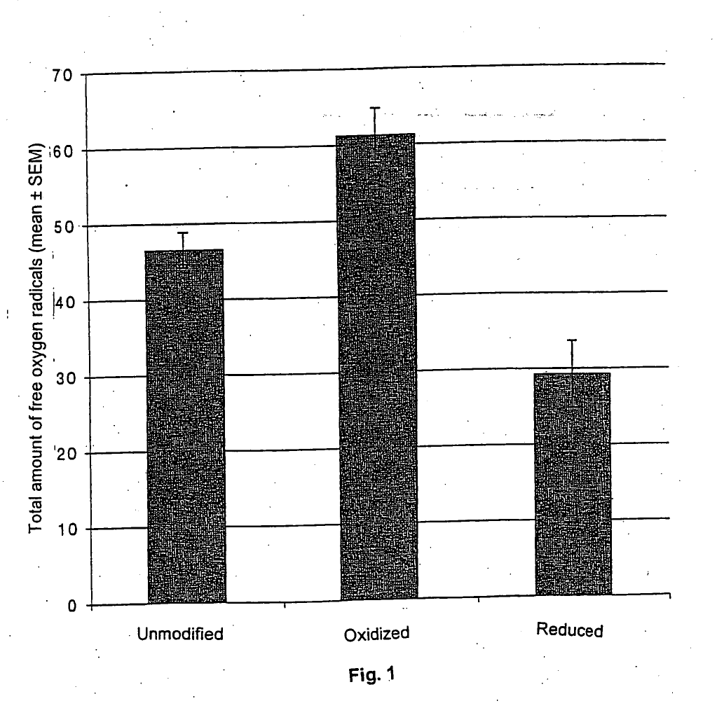

First of all leukocytes were isolated from human veinous blood and the cells were then left in contact with cotton wool compresses and stress activation of the cells was measured as the release of reactive oxygen with luminol-enhanced chemiluminiscense. The result of this measurement is shown in FIGS. 1-3.

From FIG. 1 it is apparent that the leukocytes are activated on contact with cotton wool compresses. The first bar in FIG. 1 shows the activation of an untreated cotton wool compress, the second bar the activation of a cotton wool compress which has been oxidized with periodic acid, and the third bar the activation of a cotton wool compress which has been reduced with cyanoborohydride.

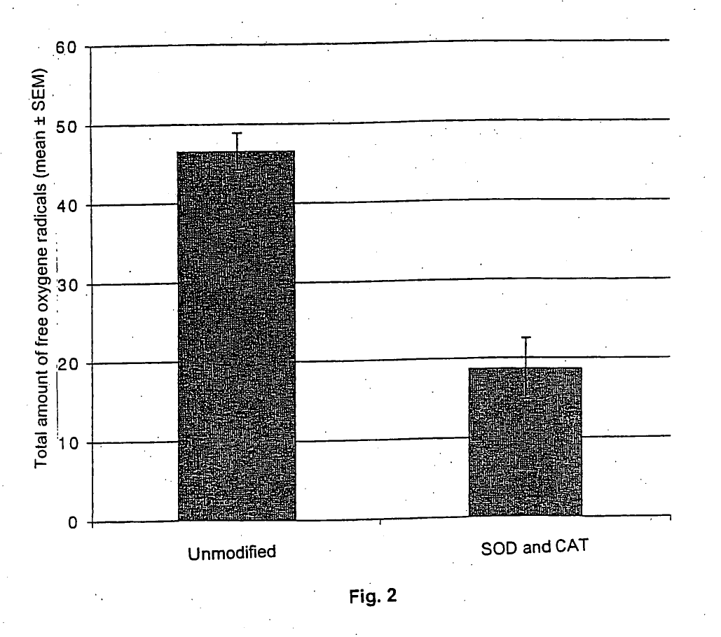

In FIG. 2 the second bar shows activation of a cotton wool compress to which two enzymes, superoxide dismutase (SOD) and catalase (CAT), have been covalently bound with the aid of a two-stage reaction where the cellulose is first oxidized with periodic acid, and the enzymes are then added. The cellulose is then reduced again with cyanoborohydride. The first bar in FIG. 2 shows the activation of an untreated cotton wool compress. As is apparent from FIGS. 1 and 2, there is a considerable decrease in the quantity of free oxygen radicals on activation with a cotton wool compress to which enzymes have been added.

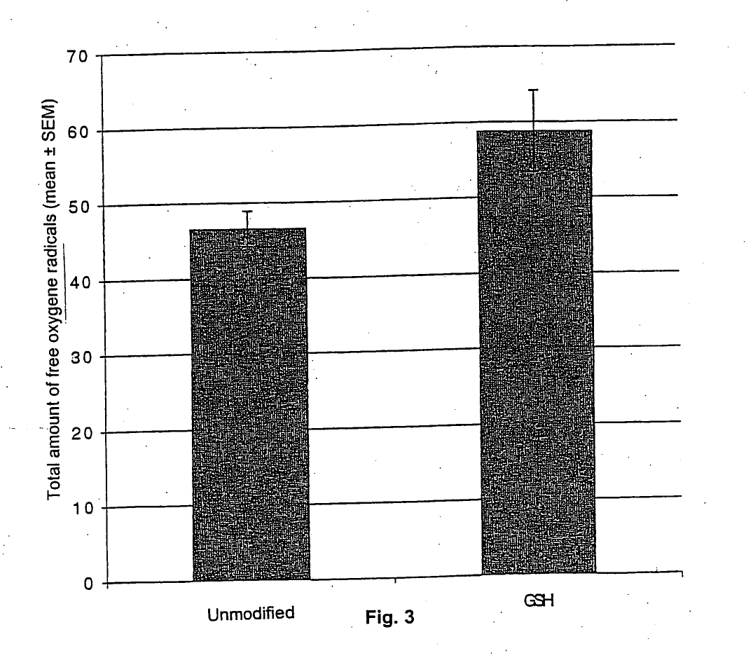

In FIG. 3 the second bar shows activation of a cotton wool compress to which a physiological saline solution with glutathione (final concentration 0.05 mM) has been added. On comparison with the first bar, which relates to the activation of an untreated cotton wool compress, it is apparent from FIG. 3 that glutathione does not affect activation of the leukocytes and that these produce a somewhat increased quantity of reactive oxygen.

It is thus apparent that unlike SOD and CAT additives the addition of glutathione does not cause any decrease in the occurrence of free oxygen radicals.

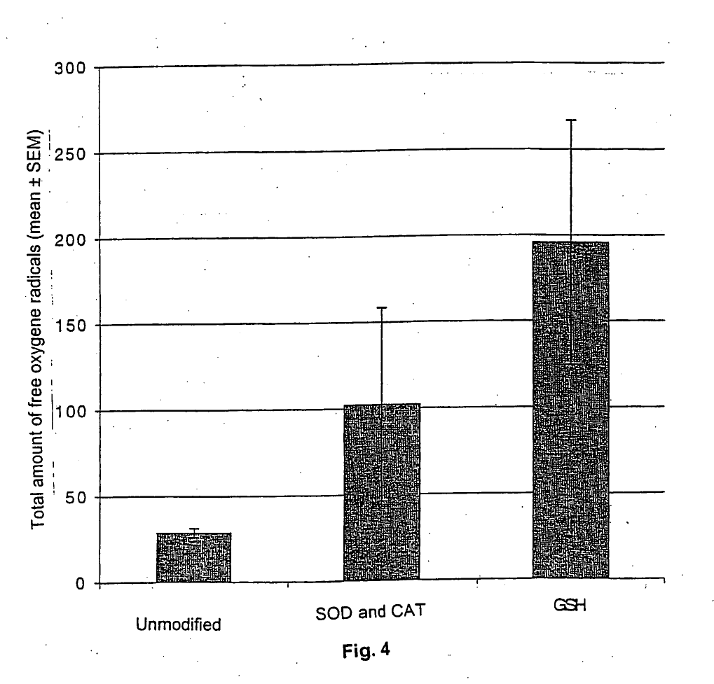

The ability of the leukocytes to react against a microbial agent after contact with the cotton wool compresses was then tested by addition of zymosan, a fungal spore used to test the ability of the leukocytes to kill microbes. The result is shown in FIG. 4. From the first bar in this figure it is apparent that the cells which have been activated by an untreated cotton wool compress have largely lost the ability to be activated by zymosan, while it is apparent from the second and third bar that the cells which have been in contact with cotton wool compresses with addition of enzymes or glutathione retain the ability to be activated by zymosan.

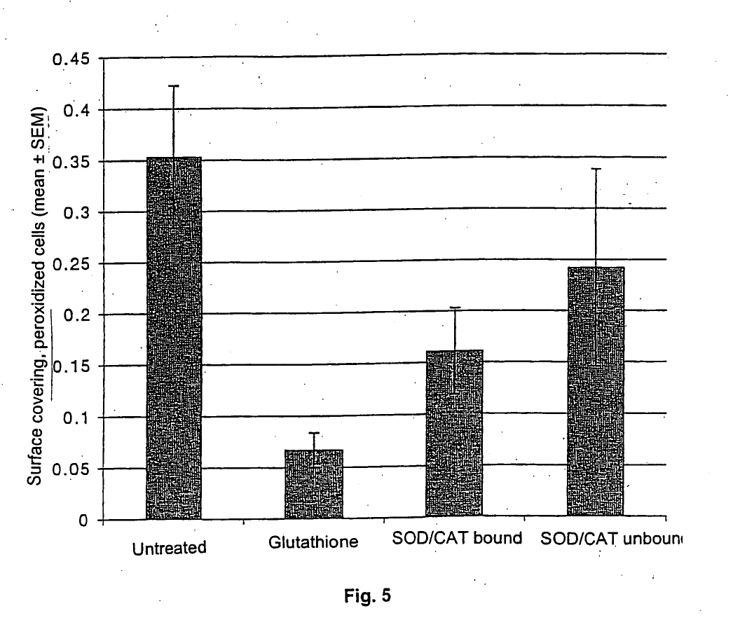

Lipid peroxidation of cell membranes during the contact between leukocytes and cotton wool compresses was measured with a fluorescent probe, diphenyl-1-pyrenylphosphine (DPPP), which reacts with membrane peroxides and forms a fluorescent oxide, see Okimoto, Watanabe, et al., 2000 FEBS Letter, vol 474, pages 137-140. The result of this measurement is shown in FIG. 5. It is apparent from this figure that both enzyme treatment and glutathione treatment reduce the lipid peroxidation of the cell membrane.

From the investigation made it is thus apparent that addition of glutathione to a wound pad in a wound bandage, unlike addition of enzymatic antioxidants, reduces lipid peroxidation of the cell membrane of the leukocytes without reducing the activability of the leukocytes. The leukocytes are thus given protection against oxygen radicals without affecting their ability to kill bacteria.

The same effect as is achieved with glutathione can be achieved with other low-molecular-weight non-enzymatic thiolic antioxidants, such as N-acetylcysteine.

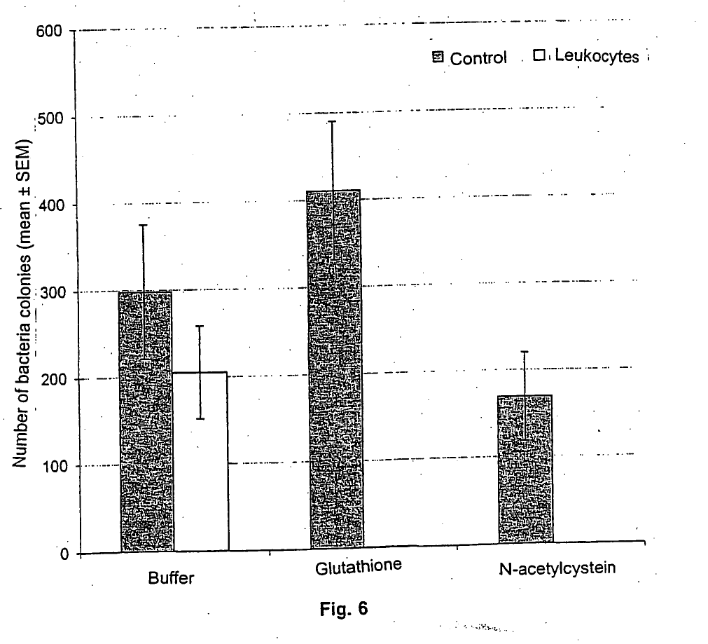

The ability of leukocytes to kill bacteria in the presence of glutathione (10 mM) or N-acetylcysteine (10 mM) in solution was studied in the following manner: Leukocytes (1×10⁵ cells/ml) and Staphylococcus aureus (1×10⁶ cells/ml) were incubated together at 37° C. for two hours. The leukocytes were killed and the remaining bacteria were allowed to grow on a blood agar plate for 24 hours, after which the number of bacterial colonies (CFU) was calculated. Control samples without leukocytes were done in parallel with all the tests. The result is shown in FIG. 6. A small number of colonies means that the leukocytes have good ability to kill bacteria. From the figure it is apparent that the leukocytes kill the bacteria completely when glutathione or N-acetylcysteine is added. The controls show that this killing effect does not depend on the ability of the additives to kill bacteria.

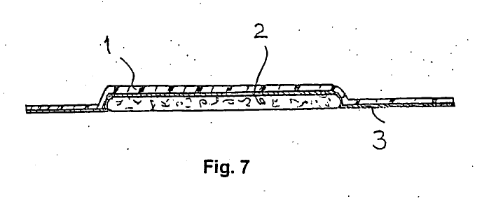

FIG. 7 shows a schematic embodiment of a wound bandage according to the invention. This wound bandage comprises a carrier layer 1, a central wound pad 2 and an adhesive coating 3.

The carrier layer 1 can for example be made up of a plastic layer, a non-woven layer or a plastic-non-woven laminate and the adhesive coating 3 can be made up of a glue of the type which is usual in a wound bandage, such as acrylate glue, or of a skin-friendly adhesive in the form of a hydrophobic or hydrophilic gel.

The wound pad 2 can consist of one or more layers of cotton fibres, cellulose fibres or other types of absorbent fibres. Absorbent foam material can also be used as material for the wound pad. According to the invention a low molecular thiolic antioxidant, such as glutathione or N-acetylcysteine, is added to the wound pad. The addition is suitably done by mixing the substance in a solution in a quantity of 0.005-5 g per litre solution, which is then left to be absorbed by the wound pad, after which this is left to dry. Another way to add one or more of the above-mentioned substances to a wound pad can be to dissolve the substance directly in a gel or other viscous solution. In a variant that is not shown of a wound bandage according to the invention the adhesive coating is made up of a gel layer which extends over the wound pad on the side thereof which is turned towards the wound when it is used. The gel layer is perforated at least within the area of the wound pad, so that the latter can suck exudate from the bed of the wound. In such a wound bandage glutathione or N-acetylcysteine, can also be added to the gel layer. It is also conceivable to add the above-mentioned substance only to the gel layer or only to the wound pad in such a wound bandage.

1. Wound bandage, characterized in that a non-enzymatic low molecular thiolic antioxidant, such as glutathione or N-acetylcysteine, is added to a layer of the wound bandage which, when the bandage is used, comes into contact with a wound.

2. Wound bandage according to claim 1, characterized in that an addition of a non-enzymatic low molecular thiolic antioxidant, such as glutathione or N-acetylcysteine, is adsorbed to a wound pad of fibre or foam material.

3. Wound bandage according to claim 1, characterized in that the bandage comprises a hydrophobic or hydrophilic gel to which a non-enzymatic low molecular thiolic antioxidant, such as glutathione or N-acetylcysteine, is added.
